(19) United States
(12) Patent Application Publication
(10) Pub. No.: US 2015/0267244 A1
(43) Pub. Date: Sep. 24, 2015

(54) Method for Detecting Endotoxins and/or 1,3-ß-D-Glucans in a Sample

(71) Applicant: LEIBNIZ-INSTITUT FÜR NEUE MATERIALIEN GEMEINNÜTZIGE GMBH, Saarbrücken (DE)

(72) Inventors: Melanie Kucki, Bernhardzell-Waldkirch (CH); Annette Kraegeloh, Blieskastel-Brenschelbach (DE)

(73) Assignee: Leibniz-Institut fuer Neue Materialien gemeinnuetzige GmbH, Saarbruecken (DE)

(21) Appl. No.: 14/433,116

(22) PCT Filed: Oct. 24, 2013
(86) PCT No.: PCT/EP2013/072328

§ 371 Date: Apr. 2, 2015

(30) Foreign Application Priority Data
Oct. 26, 2012 (DE) 10 2012 110 288.0

(51) Int. Cl. C12Q 1/56

(52) U.S. Cl. CPC C12Q 1/56; G01N 2333/195; G01N 2400/24

(57) ABSTRACT

Detection of endotoxins and/or 1,3-β-D-glucans in a sample includes making contact with the sample using an amoebocyte lysate and at least one type of particles having a surface made of at least one metal or metalloid oxide. This makes it possible to considerably increase the sensitivity of lysate detection.

### FIELD OF THE INVENTION

The invention relates to a method for detecting endotoxins and/or 1,3-β-D-glucans, to a kit for carrying out the method and to the use of particles in such a method.

### PRIOR ART

Microbiological contaminants in products can trigger severe diseases in humans. In this connection, Gram-negative bacteria in particular play an important role. For many products, for example for medical devices, it is mandatory to check for such bacteria or their decomposition products.

For the toxicity of these bacteria, an important role is played by the endotoxins. These are constituents of the cell membrane of Gram-negative bacteria. They are released especially during decomposition of the bacteria.

Different methods have been developed to detect these endotoxins. Many of the methods are based on the triggering of the coagulation cascade in the blood of horseshoe crabs (Limulidae), more particularly Limulus polyphemus and Tachypleus tridentatus, by the endotoxins. To this end, use is made of lysates of blood cells (amebocytes). The test is accordingly referred to as a Limulus amebocyte lysate test (LAL, or TAL in the case of use of Tachypleus tridentatus).

In said test, the coagulation cascade can be read in different ways. For instance, gelation of the sample can be established (gel clot test). Other methods are based on turbidity measurements or kinetic turbidity measurements. Other methods use fluorescent substrates for enzymes of the coagulation cascade.

The simplest method is the gel clot test. It is possible to apply it in many different situations and to carry it out without great expenditure in terms of apparatus. Also, it is classified as the most reliable form of detection according to the European Pharmacopoeia. The detection limit is 0.03 EU/ml (endotoxin units/ml) for most available tests.

However, a problem with this form of detection is the interference by many different substances in the sample. Therefore, it is often necessary to dilute the samples in order to reduce possible interferences. However, this also dilutes the endotoxins present in the sample. Therefore, the sensitivity of detection is frequently insufficient.

In order not to fall short of the sensitivity required for a sample, a sample may not be diluted beyond the maximum valid dilution (MVD). This is calculated from the sensitivity of detection and from the endotoxin limit predefined for the sample to be tested. As a result, a measurement is not possible for some samples, since they cannot be sufficiently diluted to avoid interferences.

Also, the gel clot test in particular requires a large amount of reagent, which must be obtained from living animals.

The coagulation cascade can also be triggered by other substrates. For instance, the described form of detection can be used to detect 1,3-β-D-glucans (1,3-beta-D-glucans) too.

### OBJECT

It is an object of the invention to provide a method which improves the sensitivity of detection for endotoxins and/or 1,3-β-D-glucans.

Solution

This object is achieved by the inventions having the features of the independent claims. Advantageous further developments of the inventions are characterized in the dependent claims. The wording of all the claims is hereby incorporated in the content of this description by reference. The inventions also encompass all meaningful and more particularly all mentioned combinations of independent and/or dependent claims.

The invention provides a method for detecting endotoxins and/or 1,3-β-D-glucans in a sample, comprising the following steps:

a) contacting the sample with an amebocyte lysate and at least one type of particles having a surface composed of at least one metal or metalloid oxide;

b) examining the sample for a change, wherein the occurrence of a change demonstrates the presence of endotoxins and/or 1,3-β-D-glucans of the endotoxin.

Individual method steps will be described in more detail below. The steps do not necessarily have to be carried out in the specified order, and the method to be depicted can also have further, nonmentioned steps.

The invention provides for the detection of endotoxins and/or 1,3-β-D-glucans. For the purposes of the invention, an endotoxin is understood to mean a pyrogenic constituent of the cell membrane of Gram-negative bacteria. Lipopolysaccharides (LPS) are concerned.

A 1,3-β-D-glucan is understood to mean any water-soluble polysaccharide or derivative thereof which can trigger the coagulation cascade in natural LAL and comprises at least two glucose molecules connected by means of a β-1,3-glycosidic linkage. The polysaccharide can also comprise yet further glycosides, which can also be linked to one another in another way.

In a first step, the sample is contacted with an amebocyte lysate and at least one type of particles. The amebocyte lysate is a lysate which is reconstituted before contacting or during contacting. Reconstitution may be necessary when the lysate is, for example, stored lyophilized. Contacting preferably consists of preparing a mixture of all the components used.

It was found that, surprisingly, the addition of the particles can distinctly increase the sensitivity of detection. This provides not only the advantage that a sample can be diluted more greatly, but also it reduces the consumption of reagent. At the same time, it might also lead to quickening of detection. In the case of the gel clot test, detection usually takes 1 hour.

The order in which the components are added is not crucial. One possibility is to first add the nanoparticles to the sample and to add the lysate only afterwards. Preferably, the lysate is added as the last constituent. The particles can also be first added to the lysate. The method can also additionally comprise the addition of further components.

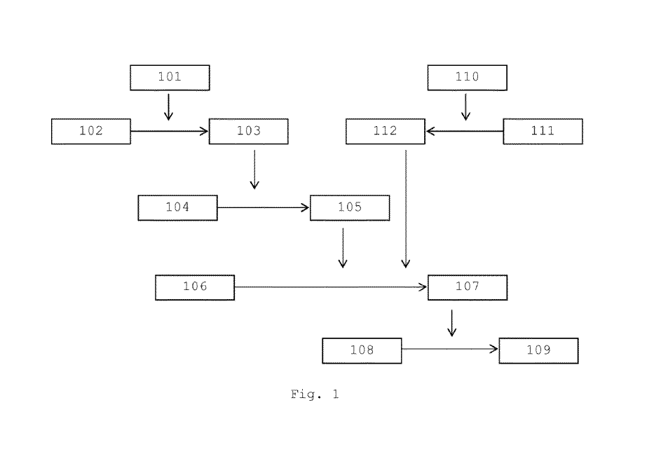

For the purposes of the invention, amebocyte lysate is understood to mean any lysate or part thereof which has been obtained from horseshoe crabs and/or prepared therefrom thereafter in vitro. The lysate can also comprise only or additionally one or more isolated or recombinant components of the coagulation cascade of the horseshoe crab. The amebocyte lysate has been preferably obtained from horseshoe crabs and/or consists of isolated or recombinant constituents of the coagulation cascade of the horseshoe crab. These are preferably the enzymes from FIG. 1.

The amebocyte lysate (AL) is preferably a lysate prepared from the hemolymph of horseshoe crabs (Limulidae), particularly preferably Limulus polyphemus, Tachypleus gigas, Tachypleus tridentatus and Carcinoscorpius rotundicauda. Preference is given to the lysate of Limulus polyphemus (LAL) and Tachypleus tridentatus (TAL).

FIG. 1 shows a diagram of the coagulation cascade in the detection of endotoxins or 1,3-β-D-glucans. An endotoxin 101 activates firstly factor C 102 into 103. This acts on a factor B (104→105: activated factor B). This activated factor B 105 activates the proclotting enzyme 106 to produce the clotting enzyme 107. This enzyme hydrolyzes specific positions of coagulogen 108 to generate coagulin 109. This leads to gelation or turbidity of the sample. This activation is also referred to as the “factor C pathway”.

The coagulation cascade can also be triggered by a different route by 1,3-β-D-glucans. In this case, such a reactive glucan (110) activates a factor G (111→112). This activates the proclotting enzyme 106 and thus leads to gelation of the sample. This activation is also referred to as the “factor G pathway”.

The lysate according to the invention can also comprise only parts of the coagulation cascade. For example, it is possible to suppress one of the two reaction pathways proceeding from endotoxins or 1,3-β-D-glucans or to remove the relevant enzymes, for example factor G, in order to avoid an interference by 1,3-β-D-glucans. Similarly, the activity of the relevant reaction pathway can be reduced. This can also be influenced by the preparation of the lysate.

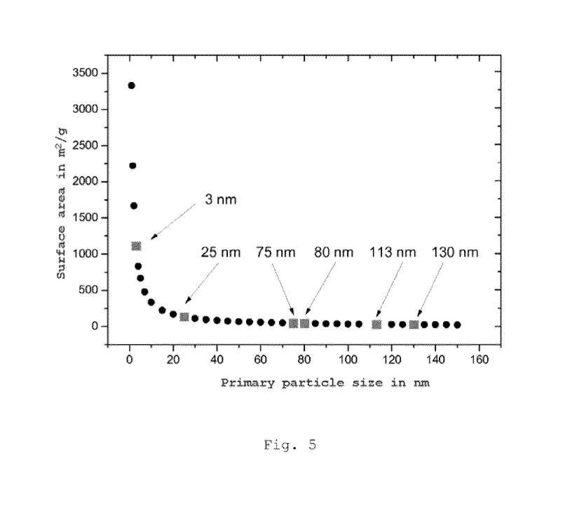

The lysate can also comprise recombinant enzymes of the coagulation cascade, for example the enzymes from FIG. 5. For example, recombinant factor C can be used. In this case, the change to be identified can be achieved by a specific substrate of the activated factor C.

The sample is contacted with at least one type of particles, preferably having a particle size of below 500 nm.

One type of particles is understood to mean particles which are alike in terms of their composition, size and morphology.

The particles can have any shapes. They can be platelet-shaped, fibrous, rod-shaped or spherical. They can be amorphous, porous or crystalline.

The particles have a surface composed of at least one metal or metalloid oxide. This means that at least part of their surface consists of at least one metal or metalloid oxide. The particles can entirely consist of a metal or metalloid oxide.

The at least one metal or metalloid oxide is preferably selected from the group comprising the oxides of Mg, Ca, Se, Ba, Al, Si, Sn, Pb, Bi, Ti, Zr, V, Mn, Nb, Ta, Cr, Mo, W, Fe, Co, Fu, Cu, Zn, Ce and Y. Preferably, the at least one metal or metalloid oxide is selected from the group containing silicon dioxide, titanium dioxide or zirconium dioxide. Preferably, it is silicon dioxide or titanium dioxide, particularly preferably silicon dioxide. In a preferred embodiment, amorphous silicon dioxide is involved.

The particles can also additionally comprise other materials, preferably inorganic substances such as oxides. For example, the materials can be support materials which are at least partly coated with the at least one metal or metalloid oxide. Different materials can be used as the support materials. The materials can be organic or inorganic materials.

In the case of organic materials, the particles or platelets can be composed of an organic polymer.

In the case of inorganic materials as support materials, the particles or platelets can be composed of oxides, sulfides, selenides, tellurides and/or phosphides. Preference is given to oxides of metal and metalloids such as, for example, Mg, Ca, Se, Ba, Al, Si, Sn, Pb, Bi, Ti, Zr, V, Mn, Nb, Ta, Cr, Mo, W, Fe, Co, Fu, Cu, Zn, Ce and Y. The particles can also be particles which contain multiple oxides. Preference is given to iron oxide particles. Particularly preferably, the support materials are iron oxide particles having a primary particle size of below 10 nm.

In a preferred embodiment, the support materials are completely coated with the metal or metalloid oxide, preferably with silicon dioxide. The particles are core/shell particles, the shell of which consists of the metal or metalloid oxide.

Coating of the support materials and preparation of the particles are preferably achieved according to the sol-gel method. This method is based on the acidic or alkaline hydrolysis of matrix formers. The matrix formers are hydrolyzable precursor compounds of the at least one metal or metalloid oxide. For example, this can be halides or alkoxides. Compounds of formula I

SiX₄ (I)

are preferably used as matrix formers, where X can be identical or different and is a hydrolyzable group, selected from the group of the halides (Cl, Br, I) or alkoxides (C₂-C₈-alkoxides). Examples of such compounds are tetramethoxysilane or tetraethoxysilane.

When using the support materials, the result is a condensation of a metal or metalloid oxide layer, preferably a silicon dioxide layer, on the surface of the support material.

If the particles are to be surface-modified, silanes having at least one nonhydrolyzable radical can be added to the reaction. These silanes are incorporated into the SiO₂ matrix which forms and remain within the matrix and on the surface of the particles.

The surface modification can also take place by means of ionic or van der Waals bonds, for example by means of interaction of the particle surfaces with carboxylic acids, amines, hydroxyl groups. Molecules, oligomers or polymers can be involved.

Preferably, the particles according to the invention do not have any or only have a slight surface modification on the metal or metalloid oxide surface. The lysate must be able to interact with the metal or metalloid oxide. A surface modification reduces the interaction between the proteins of detection and the surface composed of metal or metalloid oxide. Therefore, the surface modification leads to a lower increase in the sensitivity of detection than by unmodified particles. This is simple to determine by comparative experiments. Nevertheless, slightly modified particles are in line with the invention when they lead to an increase in the sensitivity of detection compared to detection without particles.

Preferably, the particles are noncovalently surface-modified; particularly preferably, the particles are not surface-modified.

The proportion of silanes having a nonhydrolyzable radical is therefore preferably below 1/100 000 in mol, preferably below 1/500 000 , based on the silanes used for preparing the particles or coating.

In one embodiment of the invention, less than 1% of the metal or metalloid oxide surface is modified.

In a further development of the invention, the particles have a specific surface area of over 10 m²/g, based on the metal or metalloid oxide.

In a further development of the invention, the particles have a primary particle size (measured by TEM/SEM) of below 500 nm, preferably of below 300 nm, particularly preferably of below 250 nm. In this case, the primary particle size is preferably between 2 nm and 200 nm.

In a further development of the invention, the particles are nanoparticles. These are particles which have a primary particle size (measured by TEM/SEM) of below 100 nm. Preference is given to particles having a primary particle size of below 50 nm, preferably below 10 nm. The primary particle size of the nanoparticles can also be between 2 nm and 50 nm.

The particles can also be characterized on the basis of their hydrodynamic diameter (measured in water by DLS). The hydrodynamic diameter is preferably below 500 nm, particularly preferably below 200 nm, including 200 nm. The hydrodynamic diameter is preferably between 10 nm and 200 nm.

The particles are preferably redispersible to the primary particle size. They are not present as aggregates.

Only one type of particles can be used. Alternatively, it is, however, also possible to use two, three, four or more types of particles. Preferably, all the types of particles used in the detection satisfy the requirements according to the invention.

In a further development of the invention, the particles have a zeta potential in water of below −30 mV, preferably below −40 mV. Particular preference is given to a zeta potential between −40 mV and −45 mV. The zeta potential has a particular influence on the interaction of the particles with the lysate.

The concentration of the particles in the contacted sample is preferably below 1 mg/ml. However, a distinctly lower concentration can also be selected. The concentration of the particles can have an influence on the sensitivity of detection. For instance, higher concentrations increase the sensitivity of detection. Excessively high concentrations can also adversely affect detection. The permissible concentrations can be established by means of simple comparative experiments by a person skilled in the art for the particles used. Preferably, the concentration of the particles is below 500 μg/ml, below 250 μg/ml, below 125 μg/ml or below 62.5 μg/ml. In the case of particles having a primary particle size of below 10 nm, the concentration is preferably below 100 μg/ml.

Detection is usually carried out at a pH between 6 and 8.

After contacting of the sample, the sample is examined for a change. This change can concern different properties of the sample. For instance, the consistency of the sample can change (gelation). The turbidity or the color of the sample can also change.

The change in the sample can be measured using any appropriate methods. These can be optical methods such as visual inspection following turning of the sample, turbidity, transmission, absorption or fluorescence. The measurement can take place at a particular time or continuously. For instance, the kinetics of the change in the sample can also be determined and evaluated. It is also possible to measure the fluorescence of fluorescent probes used, which indicate the activation of the coagulation cascade.

These can, for example, be substrates for individual enzymes of the coagulation cascade which are shown in FIG. 1. Such substrates can be short peptide chains which are modified with detectable probes or precursors thereof. These can, for example, be nitroanilines, which can be converted to dyes.

The presence of a change in the sample indicates the presence of endotoxins and/or 1,3-β-D-glucans. In this connection, it may be necessary, depending on the lysate used, to verify the result by control experiments, for example addition of glucan blockers, dilution series, positive controls and negative controls. This also makes it possible to distinguish between endotoxins and 1,3-β-D-glucans. For the purposes of the invention, endotoxins and/or 1,3-β-D-glucans also encompass only one type of endotoxin and/or one type of 1,3-β-D-glucan.

In a preferred embodiment of the invention, the particles are spherical silicon dioxide particles having a primary particle size between 30 nm and 150 nm. It is also possible to use mixtures of at least two particles having differing particle size.

In a further preferred embodiment of the invention, the particles are spherical metal oxide/silicon dioxide core/shell particles, preferably iron oxide/silicon dioxide core/shell particles, having a primary particle size between 2 and 100 nm.

In the case of such particles, an increase in sensitivity to 0.000875 EU/ml can be attained. This corresponds to a factor of 34 compared to standard detection.

The particles of the invention increase the sensitivity of gel clot detection for endotoxins preferably to below 0.015 EU/ml, 0.007 EU/ml, 0.0035 EU/ml or 0.00175 EU/ml.

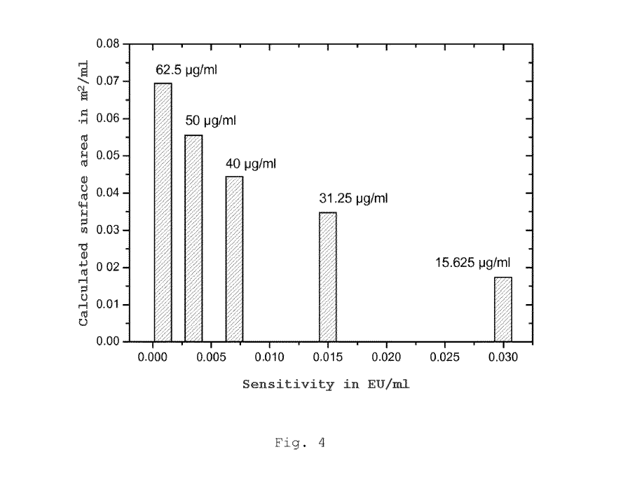

FIG. 4 shows that sensitivity increases depending on the available surface area. In one embodiment of the invention, the available calculated surface area of the particles in the detection is over 0.02 m²/ml, preferably over 0.04 m²/ml.

The method can comprise yet further steps. For instance, it may be additionally necessary to adjust the pH of the sample.

Also, it may be necessary to dilute the sample and positive controls in order to avoid interferences. These courses of action are known to a person skilled in the art from the customary procedure for lysate detection.

The method can also comprise the addition of further additives. Depending on the detection method used, these can be flocculants, detection probes (e.g., fluorescently labeled substrates for enzymes of the lysate) or buffers. This can be a substrate for the activated factor C, proclotting enzyme or clotting enzyme.

The method can also comprise carrying out control samples in order to verify the results. These can be positive samples, negative samples and/or dilution series. The method can also comprise carrying out IECs.

The invention further provides a kit for detecting endotoxins. Such a kit comprises an amebocyte lysate and at least one type of particles having a surface composed of at least one metal or metalloid oxide.

The kit is preferably a kit for carrying out the method according to the invention.

In said kit, the particles can be present in suspension or in dry form. The amebocyte lysate can be present reconstituted or in reconstitutable form.

The kit can contain yet further consitutents such as buffers or standards.

The invention also provides for the use of particles having a surface composed of at least one metal or metalloid oxide for improving the sensitivity of a detection of endotoxins with an amebocyte lysate.

The particles are preferably the particles described for the method.

Further details and features are revealed by the following description of preferred exemplary embodiments in conjunction with the dependent claims. Here, the respective features can be realized on their own or in combination with one another. The ways of achieving the object are not restricted to the exemplary embodiments. For example, indicated ranges always comprise all—nonmentioned—intermediate values and all conceivable subintervals.

### Materials and Methods

### ABBREVIATIONS

EU endotoxin unit
IEC inhibition/enhancement control
λ sensitivity of assay/detection
MVD maximum permitted dilution of a sample, the endotoxin content of which can still be reliably determined
LAL Limulus amebocyte lysate
LPS Lipopolysaccharides

1. Preparation of the Nanoparticles

The silicon dioxide particles were prepared using a modified Stober process (Stoeber et al., 1968) or by L-arginine-catalyzed hydrolysis of tetraethoxysilane (TEOS) in a biphasic water/cyclohexane system (according to Hartlen et al. 2008). The iron oxide cores for the silicon dioxide-coated particles were obtained from Nanogate AG (Quierschied-Gottelborn, Germany) and coated with silicon dioxide using a modified Stober process. One of the nanoparticles used contained a fluorescent label (f) containing the dark red fluorescent dye Atto647N-NHS. To this end, a silane linker was coupled to the dye. The thus modified dye was added to the water/cyclohexane system in the synthesis of the particles and, in this way, integrated into the silicon dioxide matrix of the particles. The modification with PEG was achieved by condensation of a mPEG750-modified silane linker on the surface of the particles. All the chemicals for the synthesis of the particles were obtained from Sigma-Aldrich (Schnelldorf, Germany). Atto647N-NHS ester (NHS: N-hydroxysuccinimide ester) was obtained from Atto-Tec (Siegen, Germany). Endorem®, a composition of dextran-coated, superparamagnetic iron oxide nanoparticles used as contrast agent for magnetic resonance imaging (MRI), was obtained from Guerbet GmbH (Sulzbach, Germany) and used as reference material. After preparation, all the particles were cleaned by means of dialysis against endotoxin-free Milli-Q water, followed by a sterile filtration on a sterile work surface with cellulose membranes having a 0.2 μm pore size. The particles were kept at 4-8° C. until they were used. The particles and their properties are specified in table 1.

2. Characterization of the Particles

For the characterization of the particles, aliquots of suspensions of sterile-filtered particles were used. Particle size and morphology were determined by scanning electron microscopy (SEM) and transmission electron microscopy (TEM). For SEM, undiluted samples of the suspensions were applied to silicon surfaces and dried under reduced pressure. The hydrodynamic diameter of the particles was determined by dynamic light scattering (DLS, Dyna Pro Titan, Wyatt Technology Europe GmbH). The zeta (ζ) potential of the particles was measured in 1 ml samples in disposable cuvettes at 25° C. using a Zetasizer Nano (Malvern, Germany). UV-visible spectra within the wavelength range of 350-800 nm were recorded using a Cary 5000 spectrometer (Varian Inc., Germany). The pH of all the samples was measured and was within the range prescribed for the assay (pH 6-8). It was therefore not necessary to adjust the pH.

3. Determination of Endotoxins

Endotoxin tests were carried out using the LAL gel clot assay from Lonza (Pyrogent™ Plus N294-03, Lonza Walkersville Inc., Walkersville, Md., USA). All the materials used were sterile and, according to the information from the manufacturer, pyrogen-free. The tests were carried out according to the protocol “Nanokon SOP 2.2.2: Detection and semi-quantification of endotoxin contaminations in nanoparticle suspensions—Limulus amebocyte lysate (LAL) gel clot assay” published on the DaNa homepage www.nanopartikel.info (http://nanopartikel.info/files/content/dana/Dokumente/Projekte/SOPs/Nanokon%20SOP%202%202%202_gesch%C3% BCtzt.pdf Kucki, 2012). This protocol is based on the information from European Pharmacopoeia, monograph 2.6.14. Bacterial Endotoxins (European Pharmacopoeia, 2005), ISO 29701 (ISO 29701, 2010) and (Dobrovolskaia and Neun, 2011).

The manufacturer-specified sensitivity of the LAL gel clot assay (0.03 EU/ml; endotoxin unit per milliliter) was checked according to the information from the manufacturer and confirmed for all the batches used.

For suspensions of nanoparticles, a value of 0.5 EU/ml was adopted as the endotoxin limit. A sensitivity of detection (λ) of 0.03 EU/ml and a sample concentration of 1 mg/ml gives rise to a maximum valid dilution (MVD) of 16 (MVD=sample concentration*endotoxin limit/sensitivity of detection (λ)).

Dilutions and controls were carried out using endotoxin-free water (LAL reagent water W50, Lonza Walkersville Inc., USA). In the event of endotoxins being detected in the samples, the samples were additionally tested for a possible disruption of the detection by 1,3-β-D-glucan. To this end, β-glucan blocker was added (Beta-G-Blocker Kit N190, Lonza Walkersville Inc., USA).

The influence of the nanoparticles on the sensitivity of the assay was also examined by IECs (inhibition/enhancement controls). This was carried out according to the aforementioned SOP and the information from the manufacturer. Particle suspensions tested as being endotoxin-free were admixed with a reference standard (certified reference standard endotoxin CSE Escherichia coli 055:B5, Lonza Walkersville Inc., USA) and the samples tested.

All the measurements were repeated with independent dilution series.

4. Exemplary Performance of the Method

4.1 Reconstitution of LAL

Lyophilized LAL was reconstituted in endotoxin-free water and swirled for at least 30 seconds (not shaken). The reconstituted LAL was used as soon as possible.

4.2 Detection of Endotoxins (Test Procedure)

All the samples, standards or controls were mixed 1:1 (vol:vol) with reconstituted LAL (100 μl in each case) and heated in a noncirculating water bath at 37° C. for 1 hour. During this heating, the samples were not moved. Thereafter, the samples were carefully turned by 180°. A solid clotted mass following turning indicates a positive test result. In the event of a negative reaction, there is no clotted mass, but instead only a liquid.

4.3 Check of the Sensitivity of Detection

Firstly, a solution containing 1 EU/ml endotoxin was used to prepare a duplicate dilution series according to the manufacturer-specified LAL sensitivity (λ) (2λ, 1λ, 0.5λ, 0.25λ). In addition, a negative control containing water (LAL reagent water) was produced.

The samples were tested according to the test procedure. The geometric mean of sensitivity was formed from the results. (

$$log {x}_{geom} = \frac{1}{n} {\sum }_{i = 1}^{n} {x}_{i}$$

where xᵢ=sensitivity; n=number of replicates of detection). The calculated geometric mean should correspond to the manufacturer-specified sensitivity.

4.4 Testing of the Nanoparticle Suspensions

A duplicate dilution series (½, ¼, ⅛, 1/16) was prepared from the suspension to be tested (1 mg/ml). A positive control containing 2λ (0.06 EU/ml) was prepared from the 1 EU/ml endotoxin solution. Endotoxin-free water (LAL reagent water) was used as negative control. The samples were tested according to the test procedure.

The concentration of endotoxins in the sample was calculated from the positive sample having the highest dilution factor (concentration=sample*λ).

If no sample was positive, it was assumed that the endotoxin concentration was below the sensitivity of the assay.

4.5 Testing of the Influence of the Nanoparticles on the Assay (IEC)

In order to test the influence of the nanoparticles on the assay, the sensitivity of the assay was tested according to the aforementioned procedure for different concentrations of the nanoparticles. For this purpose, a duplicate dilution series was prepared from a 1 EU/ml endotoxin solution (2λ, 1λ, 0.5λ, 0.25λ). In this procedure, the suspension of the nanoparticles was used for dilution, rather than endotoxin-free water. Thereafter, sensitivity was tested as described under point 4.3.

In the customary method, IEC is also prescribed in order to minimize incorrect measurements owing to inhibition or amplification effects. According to the SOP, the test is classified as uninfluenced when the measured sensitivity is within the range of 0.5λ and 2λ (IEC criteria). IECs were carried out at MVD and at least one further particle concentration.

4.6 1,3-β-D-Glucan Interference Test

If endotoxins were established, a 1,3-β-D-glucan interference test was carried out in order to rule out an interference by 1,3-β-D-glucan.

To this end, a sample of nanoparticles having twice the test concentration was prepared. β-G-Blocker (Lonza) was added thereto in a 1:1 ratio. The sample was tested as indicated above. The controls used were samples of nanoparticles without β-G-Blocker, β-G-Blocker in endotoxin-free water, positive sample with endotoxin (2λ) and β-G-Blocker.

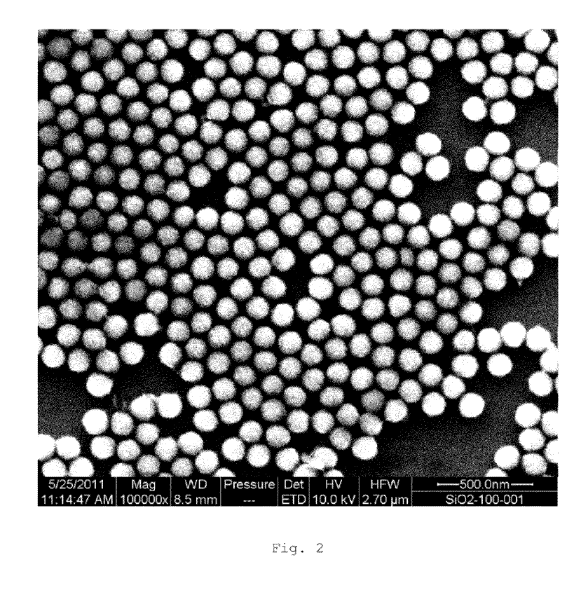

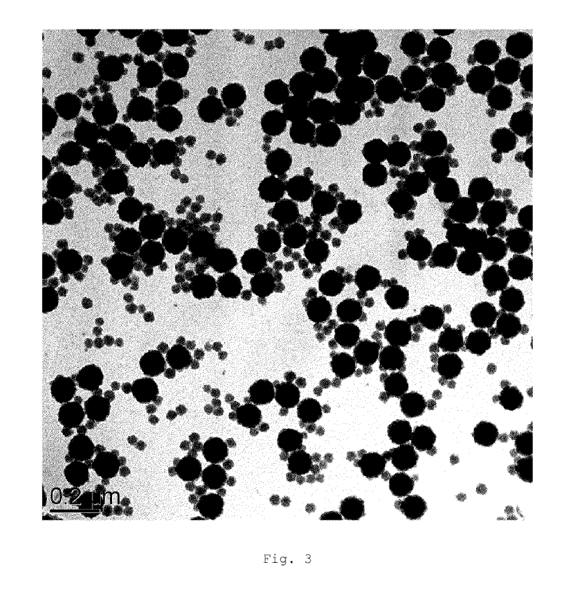

5. Experiments
FIG. 2 REM image of monodisperse silicon dioxide particles (Silica-4-130);
FIG. 3 TEM image of Silica-2; mixture of particles having two sizes: small nanoparticles having a 42 nm primary particle size (72%) and large nanoparticles having a diameter of about 108 nm (28%);
FIG. 4 Sensitivity of the LAL gel clot assay depending on the concentration used of iron oxide/silicon dioxide particles FeₓO_{y}@SiO₂-1 and the calculated surface area in m²/ml. The manufacturer-specified sensitivity of the assay is 0.03 EU/ml.
FIG. 5 Calculated surface area of the particles as a function of the primary particle size.

Table 1 shows the tested nanoparticle suspensions. In table 2, the results of testing these suspensions for nanoparticles are displayed. Although endotoxins having a content of >0.03 EU/ml were established in the case of some samples, the IEC at MVD showed in the case of almost all the samples an amplification of the sensitivity of detection. In the case of the positive samples, it was possible to demonstrate that there was no β-glucan interference. It was possible to rule out a contamination of the samples with endotoxins owing to the preparation of the nanoparticles and the control experiments.

The values between brackets in table 1 are not permissible measured values compared to a detection without nanoparticles, since in their case the IEC did not meet the specified acceptance criteria. This shows that the sensitivity of detection was especially greatly increased by the addition of these nanoparticles.

5.1 Silicon Dioxide Particles

When samples having a concentration of 62.5 μg/ml (this corresponds to MVD for nanoparticles) were measured, only Endorem and Silica-1-25 showed no change in the sensitivity of detection. However, the Silica-1-25 particles also had a distinctly altered zeta potential of −24.2 mV. Four of the tested silicon dioxide particles (Silica-2, Silica-3-80, Silica-5^{f}, Silica-6+PEG^{f}) showed an amplification of sensitivity, but within the limits of the IEC. Only Silica-4-130 (see also FIG. 2), a suspension comprising monodisperse silicon dioxide particles having a primary particle size of 130 nm, showed an amplification higher than permissible according to the IEC, when it was tested within the permissible dilution range (up to MVD).

Concentration-dependent IECs were carried out using Silica-3-80. These yielded an improvement in the sensitivity of detection to 0.015 EU/ml for all the tested particle concentrations (500 μg/ml, 250 μg/ml, 125 μm/ml, 62.5 μg/ml). However, no change in sensitivity with regard to the amount of particles used was established.

Table 3 shows concentration-dependent IECs for Silica-2 (FIG. 3). In said table, + means a positive detection and − means a negative detection. In this case, a lowering of the concentration of the particles also led to a worsening of the sensitivity of detection.

In order to test the influence of surface modification, IECs were carried out using Silica-5^{f} and Silica-6+PEG^{f}. These particles have a similar size. It was found that, in the case of a high particle concentration (500 μg/ml SiO₂), the amplification of sensitivity was different. PEG-modified particles Silica-6+PEG^{f} exhibited a lower amplification within the IEC acceptance criteria. By contrast, the amplification in the case of Silica-5^{f} was beyond the acceptance criteria. This indicates that the amplification is reduced by surface modification of the particles with organic groups.

5.2 Iron Oxide/Silicon Dioxide Core/Shell Particles

The iron oxide/silicon dioxide core/shell particles FeₓO_{y}@SiO₂-1, FeₓO_{y}@SiO₂-2^{f} and FeₓO_{y}@SiO₂-3^{f} were prepared using the same method. In the case of the fluorescently labeled particles, only one fluorescent dye was incorporated into the particles as already described. All the particle suspensions exhibited similar behavior in the LAL gel clot assay. No differences were established on the basis of the fluorescent labeling.

In the case of high concentrations (1 mg/ml and 500 μg/ml), a complete inhibition of detection was established (no coagulation). Also, it was possible to observe a precipitation of the particles. In the case of medium concentrations (250 μg/ml and 125 μg/ml), this was not observed. Instead, a brownish clotted mass formed. At MVD (62.5 μg/ml), the test was negative as expected. The 0.25 EU/ml calculated content of endotoxin was below the endotoxin limit of 0.5 EU/ml. But the IEC for all the particles showed a distinct amplification of sensitivity beyond the IEC criteria.

Table 4 shows concentration-dependent IECs for FeₓO_{y}@SiO₂-l. The results show a distinct amplification of sensitivity depending on the concentration of the particles. At a concentration of 62.5 μg/ml, it was still possible to detect 0.000875 EU/ml. This corresponds to an increase by a factor of 34.

The larger particles FeₓO_{y}@SiO₂-4^{f} were also tested. They too exhibited a distinct amplification of sensitivity of detection up to the MVD. However, it was not possible to observe a complete inhibition of the assay at 500 μg/ml.

5.3 Endorem®

Endorem® is a composition composed of superparamagnetic iron oxide nanoparticles (SPION) having a primary particle size of 5 nm. It is authorized as a contrast agent for investigating liver metastases. The particles are surface-modified with dextran.

In the tests, it was not possible to detect any endotoxins. Also, Endorem® exhibited no interaction at all with the LAL gel clot assay at the lowest concentration used (62.5 μg/ml). Even when it was used undiluted (11.2 mg/ml), a complete inhibition of detection was not observed. A precipitation of the particles was also not observed.

Numerous modifications and further developments of the described exemplary embodiments are realizable.

TABLE 1
Physicochemical properties of the tested nanoparticle suspensions

| | | | | Zeta |
|---|---|---|---|---|
| | | | Primary | potential |
| | | Hydrodynamic | particle | (mV), |
| | | diameter in | size in | 25° C., in |
| Particle | Synthesis | nm (DLS) | nm | water |
| Endorem ® | — | 246 | 5 | −35 |
| FeₓO_{y}@SiO₂-l | Stöber | 16.6 ± 5.0 | 3 | −44.7 |
| FeₓO_{y}@SiO₂-2^{f} | Stöber | 22.1 ± 6.6 | 3 | −42.9 |
| FeₓO_{y}@SiO₂-3^{f} | Stöber | 88.9 ± 49 | 3 | −43.2 |
| FeₓO_{y}@SiO₂-4^{f} | Stöber | 71.1 ± 10 | 75 | −41.4 |
| Silica-1-25 | Hartlen | 20.8 ± 2.6 | 24.6 ± 3.4 | −24.2 |
| Silica-2 | Hartlen | 97.6 ± 39 | 42 and 108 | −42.9 |
| Silica-3-80 | Hartlen | 84.2 ± 6.7 | 80 ± 5 | −42.3 |
| Silica-4-130 | Hartlen | 141.4 ± 15 | 128.3 ± 11.0 | −40.5 |
| Silica-5^{f} | Stöber | 104.0 ± 35 | 113 ± 14 | −43.2 |
| Silica-6 + PEG^{f} | Stöber | 96.0 ± 34 | 113 ± 14 | −41.2 |

^{f}with fluorescent dye

TABLE 2
Results of the endotoxin tests

| | Endotoxin in | | |
|---|---|---|---|
| | EU/ml at 1 | | |
| | mg/ml | | IEC criteria |
| Particle | particles | IEC at MVD | fulfilled? |
| Endorem ® | <0.03 | Specific | + |
| | | sensitivity | |
| FeₓO_{y}@SiO₂-l | [0.25] | Amplification | − |
| FeₓO_{y}@SiO₂-2^{f} | [0.25] | Amplification | − |
| FeₓO_{y}@SiO₂-3^{f} | [0.25] | Amplification | − |
| FeₓO_{y}@SiO₂-4^{f} | [0.25] | Amplification | − |
| Silica-1-25 | <0.03 | Specific | + |
| | | sensitivity | |
| Silica-2 | <0.03 | Amplification | + |
| Silica-3-80 | <0.03 | Amplification | + |
| Silica-4-130 | [0.06] | Amplification | − |
| Silica-5^{f} | <0.03 | Amplification | + |
| Silica-6 + PEG^{f} | <0.03 | Amplification | + |

[ ] = measured values not acceptable, since IEC criteria not fulfilled

TABLE 3
Concentration-dependent IECs for Silica-2

| Particle | IEC endotoxin value in EU/ml | | | | |
|---|---|---|---|---|---|
| concentration | | | | | Negative |
| in μg/ml | 0.03 | 0.015 | 0.007 | 0.0035 | control |
| 1000 | + | + | + | + | − |
| 500 | + | + | + | + | − |
| 250 | + | + | + | − | − |
| 125 | + | + | − | − | − |
| 62.5 | + | + | − | − | − |

TABLE 4
Concentration-dependent IECs for FeₓO_{y}@SiO2-l

| Particle | | | | | | |
|---|---|---|---|---|---|---|
| concen- | | | | | | |
| tration | IEC endotoxin value in EU/ml | | | | | |
| in μg/ml | 0.03 | 0.015 | 0.007 | 0.0035 | 0.00175 | 0.000875 |
| 62.5 | + | + | + | + | + | + |
| 50 | + | + | + | + | − | ND |
| 40 | + | + | + | − | − | ND |
| 31.25 | + | + | − | − | − | − |
| 15.625 | + | − | − | ND | ND | ND |

ND = not determined

### LITERATURE CITED

STOEBER, W., FINK, A. & BOHN, E. 1968. Controlled Growth of Monodisperse Silica Spheres in the Micron Size Range. Journal of Colloid and Interface Science, 26, 62-69.
HARTLEN, K. D., ATHANASOPOULOS, A. P. T. & KITAEV, V. 2008. Facile Preparation of Highly Monodisperse Small Silica Spheres (15 to >200 nm) Suitable for Colloidal Templating and Formation of Ordered Arrays. Langmuir, 24, 1714-1720.

### REFERENCE SIGNS

101 endotoxin
102 factor C
103 activated factor C
104 factor B
105 activated factor B
106 proclotting enzyme
107 clotting enzyme
108 coagulogen
109 coagulin
110 1,3-β-D-glucan or other LAL-activating glucan
111 factor G
112 activated factor G

1. A method for detecting endotoxins and/or 1,3-β-D-glucans in a sample, comprising:
contacting the sample with an amebocyte lysate and at least one type of particles having a surface composed of at least one metal or metalloid oxide; and
examining the sample for a change, wherein the occurrence of a change indicates the presence of endotoxins and/or 1,3-β-D-glucans.

2. The method as claimed in claim 1, wherein the particles have a zeta potential in water of below −30 mV.

3. The method as claimed in claim 1, wherein the particles have a primary particle size of below 500 nm.

4. The method as claimed in claim 1, wherein the particles in the contacted sample are present in a concentration of below 1 mg/ml.

5. The method as claimed in claim 1, wherein the metal or metalloid oxide is selected from the group containing silicon dioxide, titanium dioxide, and zirconium dioxide.

6. The method as claimed in claim 5, wherein the metal or metalloid oxide is silicon dioxide.

7. The method as claimed in claim 1, wherein the amebocyte lysate is obtained from horseshoe crabs and/or consists of isolated or recombinant constituents of the coagulation cascade of the horseshoe crab.

8. The method as claimed in claim 1, wherein the change in the sample is established by optical methods.

9. A kit for detecting endotoxins and/or 1,3-β-D-glucans, comprising an amebocyte lysate and at least one type of particles having a surface composed of at least one metal or metalloid oxide.

10. The kit as claimed in claim 9, wherein the at least one type of particles have a zeta potential in water of below −30 mV.

11-12. (canceled)

13. A method, comprising:
contacting a sample with an amebocyte lysate and at least one type of particles having a surface composed of at least one metal or metalloid oxide; and
improving a sensitivity of a detection of endotoxins and/or 1,3-β-D-glucans in the sample.

14. A method according to claim 13, wherein the at least one type of particles have a zeta potential in water of below −30 mV.
